(19) United States
(12) Patent Application Publication
(10) Pub. No.: US 2005/0124970 A1
(43) Pub. Date: Jun. 9, 2005

(54) Luer cap with antibacterial property

(75) Inventors: David Kunin, North Wales, PA (US); Joseph E. Maenner, Downingtown, PA (US)

Correspondence Address: MONTE & MCGRAW, PC, 4092 SKIPPACK PIKE, P.O. BOX 650, SKIPPACK, PA, 19474, US

(73) Assignee: Medical Components, Inc., Harleysville, PA (US)

(21) Appl. No.: 11/004,770

(22) Filed: Dec. 3, 2004

Related U.S. Application Data
(60) Provisional application No. 60/527,260, filed on Dec. 5, 2003.

(51) Int. Cl. A61M 31/00

(52) U.S. Cl. 604/508; 604/265; 604/533; 604/538

(57) ABSTRACT

A luer fitting is disclosed. The luer fitting includes a body having a first end and a second end, wherein the first end comprises an antibacterial component disposed therewith. The luer fitting is connected over a catheter that has been primed with a locking solution. The antibacterial component is fluidly engaged with the locking solution such that the antibacterial component diffuses through the locking solution. A method of reducing antibacterial activity in a catheter utilizing the fitting is also disclosed.

### CROSS REFERENCE TO RELATED APPLICATION

The present application claims the benefit of U.S. Provisional Patent Application Ser. No. 60/527,260, filed on 5 Dec. 2003.

### BACKGROUND OF THE INVENTION

Catheters are used to provide repeated access to a vascular system in a patient. A catheter may be used to infuse a fluid, such as a medication or a saline solution, or withdraw a fluid, such as blood. Multiple lumen catheter assemblies, such as those used for hemodialysis, include at least one catheter for the withdrawal of blood from a blood vessel to be sent for processing in a hemodialysis machine, while another catheter returns the blood to the vessel after processing.

Catheters may be inserted into a patient for acute care, where the catheter is intended to be in the patient for a short duration, such as for only a few days, or for chronic care, where the catheter is intended to be in the patient for a much longer duration, such as over several months.

One of the biggest obstacles to catheterization of a patient is the risk of infection. It is important to maintain sterility in and around the catheter to reduce the risk of infection. One way to maintain sterility around the exterior of the catheter in between uses is to swab the dermal area around the catheter and the exposed portions of the catheter itself with a disinfectant, such as iodine or alcohol, depending on the material used to fabricate the catheter. However, it is possible for pathogens to grow within the catheter, which may cause infections within the vascular system of the patient.

Various locking solutions have been used inside the catheter to prevent clotting within the catheter in between uses. However, heparin, the most popular anti-coagulant that is used as a locking solution, is at best, minimally successful as an antibacterial agent. It would be beneficial to be able to impart antibacterial properties into a catheter lock solution to minimize the risk of infection within the catheter in between uses.

### BRIEF SUMMARY OF THE INVENTION

Briefly, the present invention provides a luer fitting. The luer fitting comprises a body having a first end and a second end, wherein the first end comprises an antibacterial component disposed therewith.

The present invention also provides a luer fitting comprising a body having a first end and a second end. The first end includes a stylet extending therefrom. The stylet includes an antibacterial component disposed on an exterior thereof.

Additionally, the present invention also provides a method of reducing antibacterial activity in a catheter comprising: providing a catheter having a lumen and a connector connected thereto; inserting a lock solution into the lumen; releasably affixing a luer fitting to the connector, wherein the luer fitting includes an antibacterial component; and exposing the antibacterial component to the lock solution, wherein the antibacterial component disperses through at least a portion of the lock solution.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a perspective view of a luer cap according to a first embodiment of the present invention.

FIG. 2 is an end view of the luer cap of FIG. 1.

FIG. 3 is a sectional view of the luer cap taken along line 3-3 of FIG. 2.

FIG. 4 is a sectional view of the luer cap connected to a catheter lumen.

FIG. 5 is a perspective view of a luer cap according to a second embodiment of the present invention.

### DETAILED DESCRIPTION OF THE INVENTION

In the drawings, like numerals indicate like elements throughout. Certain terminology is used herein for convenience only and is not to be taken as a limitation on the present invention. The terminology includes the words above specifically mentioned, derivatives thereof, and words of similar import. The following describes a preferred embodiment of the invention. However, it should be understood based on this disclosure, that the invention is not limited by the preferred embodiment described herein.

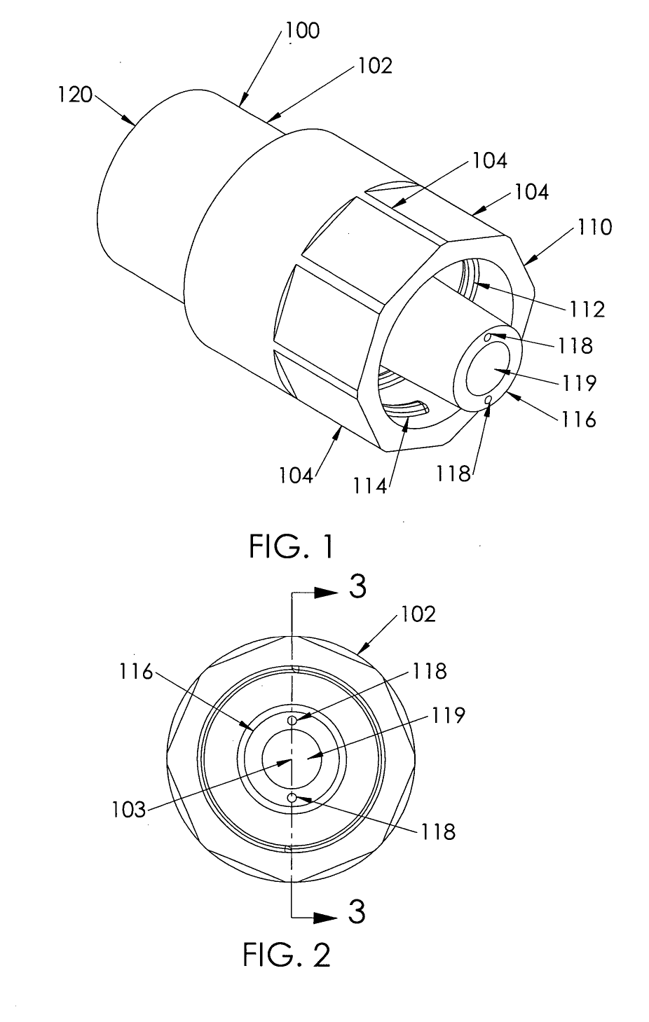

Referring to FIGS. 1-3, a luer cap 100 according to a first embodiment of the present invention is shown. The luer fitting 100 includes a preferably generally cylindrical body 102 that includes an open first end 110 a closed second end 130, and a longitudinal axis 103 extending therethrough. The body 102 includes a plurality of flats 104 disposed about the open first end 110. The flats 104 generally provide surfaces for a user (not shown) to grip the luer fitting 100 to twist the luer fitting 100 onto or off of a female luer connection (not shown).

The first end 110 includes a generally cylindrical opening 112. The interior of the cylindrical opening 112 preferably includes a female threaded connection 114. A tapered male luer connection 116 extends through the cylindrical opening 112 along the longitudinal axis 103. The luer connection 116 includes a silver matrix 118 that is embedded in the luer connection 116, with a portion of the silver matrix 118 being in fluid communication with the exterior of the luer connection 116. While a silver matrix 18 is preferred, those skilled in the art will recognize that other metals, such as gold, titanium, platinum, or other suitable metal may be used, alone or in combination with the remaining metals, in a matrix form. As an additional alternative, a matrix containing a lyophilyzed antimicrobial agent embedded therein may be used. The matrix may be a filament, a polymer, such as polyurethane, or some other suitable, biocompatible material.

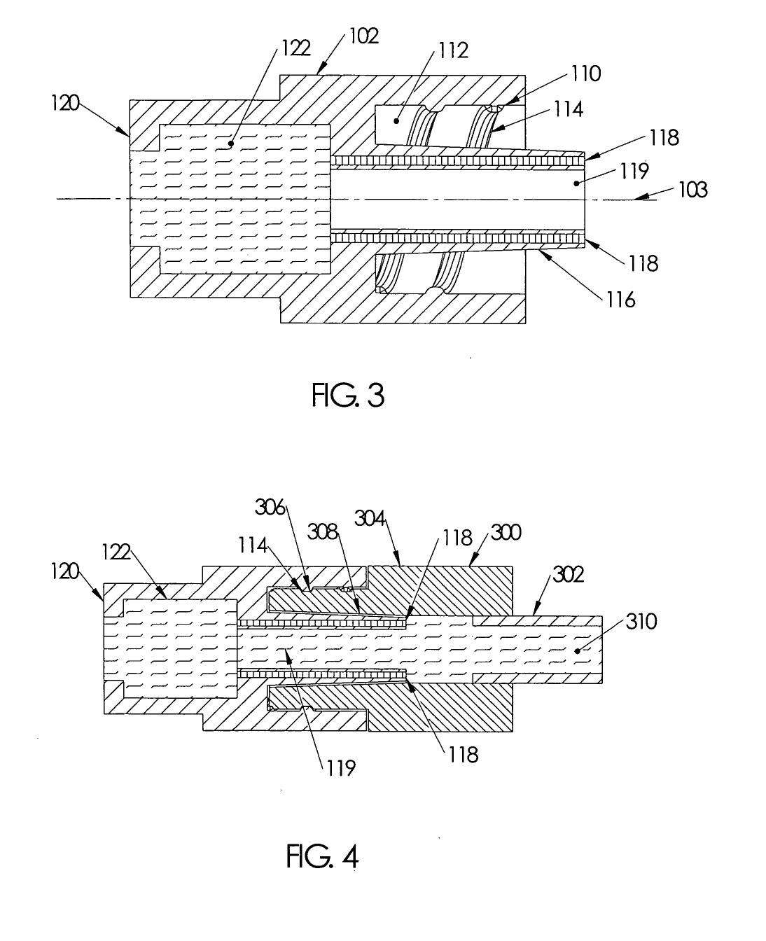

While two pieces of the silver matrix 118 are shown in FIGS. 1 and 2, those skilled in the art will recognize that more or less than two pieces of the silver matrix 118 may be incorporated into the luer fitting 100. The silver matrix 118 may be in the form of silver coated nylon filaments or some other known method of providing silver in a matrix. The luer connection 116 also includes a passage 119 that extends into the body 102 toward the closed second end 130.

The second end 130 includes a seal 122 that closes the second end 130. The seal 122 fluidly communicates with the passage 119 so that a needle (not shown) may be inserted into the seal 122 from the second end 130 and pushed through the seal 122 to the passage 119.

Preferably, the body 102 and the male luer connection 116 are both constructed from a polymer, such as polyvinylchloride, polystyrene, polypropylene, or other suitable materials. The seal 122 is preferably constructed from a fluid impermeable material that may be relatively easy to pierce with a needle and is self-sealing, such as latex or silicone.

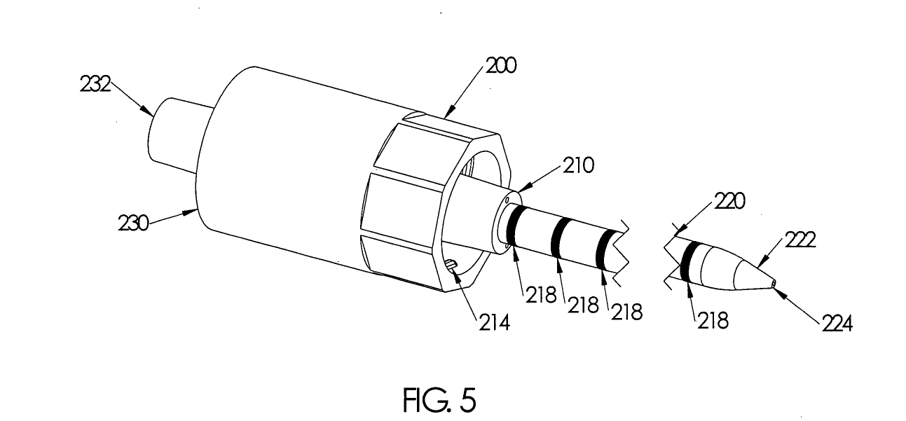

In use, the luer fitting 100 is connected to a separate device, such as a hemodialysis catheter 300, as shown in FIG. 4. Preferably, the catheter 300 includes a lumen 302 having a connector 304 fixedly connected to a proximal end of the lumen 302. The connector 304 includes male threads 306 that are sized to threadingly engage the female threaded connection 114 and a tapered female luer connector 308 that frictionally engages the male luer 116 on the luer fitting 100.

Preferably, the catheter 300 is inserted into a patient so that a distal end of the lumen 302 is inserted into the patient, such as in a blood vessel. While a preferred blood vessel is the internal jugular vein, and the catheter 300 is used for hemodialysis, those skilled in the art will recognize that the catheter 300 may be used for other applications and may be inserted in other locations in the patient.

Prior to connecting the luer fitting 100 to the catheter 300, a locking solution 310, such as heparin, alcohol, or other anticoagulant compound is injected into the lumen 302. The locking solution 310 must be biocompatible with the silver matrix 18, and allow silver ions to diffuse through the locking solution 310. Such a preferred locking solution 310 includes heparin, water, alcohol, or other suitable fluid.

The luer fitting 100 is releasably connected to the catheter 300 by inserting the male luer connection 116 into the female luer connection 308 and engaging the female threads 114 of the luer fitting 100 with the male threads 304 of the catheter 300. The locking solution 310 backflows into the luer fitting 100 due to hydrostatic pressure of the blood in the vessel, putting the silver matrix 118 into fluid communication with the locking solution 310. Silver ions diffuse at least partially into the lock solution 310 in sufficient quantity, preferably greater than approximately 30 micrograms/milliliter, to provide antibacterial effectiveness against all nosocomial pathogens. Preferably, the luer fitting 100 is connected to the catheter 300 only between dialysis treatments, which tend to take place within a 96 hour timeframe. When the luer fitting 100 is removed to perform dialysis, a replacement luer fitting 100 with a fresh silver matrix 118 replaces the original luer fitting 100 after the hemodialysis treatment is completed, thus providing a sustained concentration of silver ions in the lock solution 310 between hemodialysis treatments. IT is believed by the inventors that the silver matrix 118, in fluid communication with the locking solution 310, will provide at least 20 micrograms per milliliter of lethality over the 96 hour timeframe against nosocomial pathogens.

Referring now to FIG. 5, a second embodiment of a luer cap 200 according to the present invention is shown. The luer cap 200 includes a first end 210 having a male luer connector and female threads 214, similar to the luer cap 100 described above, but with an added antibacterial component, such as a silver matrix 218, extending distally from the first end 210. The silver matrix 218 may be embedded in a stylet 220 that is fixedly connected to the luer cap 200. Preferably, the stylet 220 is generally an elongate tube with a preferably tapered distal end 222. The stylet 220 is manufactured from a polymer, such as nylon, or a thermoplastic polyurethane elastomer, such as PELLETHANE®, or some other suitable, biocompatible material. Also preferably, a passage 224 extends longitudinally through the stylet 220, with the passage fluidly communicating with a second end 230 of the luer cap 200 that is open.

The luer cap 200 also includes a threaded luer fitting 232 disposed at the second end 230 of the luer cap 200. The luer fitting 232 allows a standard luer cap or the luer cap 100 to be threadingly engaged with the luer fitting 232. The passage 224 also extends through the luer fitting 232 so that fluid may communicate between the first end 210 and the second end 230 of the luer cap 200. Optionally, although not shown, the second end 230 of the luer cap 200 may be sealed with a fluid impermeable material that may be relatively easy to pierce with a needle and is self-sealing, such as latex or silicone.

In between hemodialysis treatments, the catheter lumen 302 is primed with the locking solution 310. The stylet 220 on the luer cap 200 is then inserted into the proximal end of the catheter 300 and at least partially through the catheter lumen 302. The female threads 214 on the luer cap 230 are threadingly connected to the male threads 304 on the proximal end of the catheter 300, as is well known in the art. In between catheter uses, when the catheter 300 is primed with the locking solution 310, the silver matrix on the stylet 220 diffuses into the locking solution 310 to provide antibacterial protection throughout the length of the catheter 300.

While the silver matrix 218 shown in FIG. 5 is shown as bands spaced along the length of the stylet 220, those skilled in the art will recognize that the silver matrix 218 may include additional and/or wider bands, or may be disposed along the entire length of the stylet 220. Further, the stylet 220 is preferably sufficiently long so as to extend a significant distance into a catheter 300 when the luer cap 200 is connected to the catheter 300. Preferably, the silver matrix 218 is disposed on the stylet 220 to provide greater than 25 mcg/ml of silver diffusion into the locking solution for a period of approximately 72 to 96 hours.

The silver matrix 218 may be disposed on the stylet 220 by adhesive, by bonding the silver matrix 218 to a polymer substrate and then ultrasonically welding the polymer substrate to the stylet 220, by chemical vapor deposition or by some other method known to those skilled in the art for depositing a metal onto a surface.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A luer fitting comprising a body having a first end and a second end, wherein the first end comprises an antibacterial component disposed therewith.

2. The luer fitting according to claim 1, wherein the antibacterial component comprises silver.

3. The luer fitting according to claim 2, wherein the silver is a silver filament.

4. The luer fitting according to claim 1, wherein the first end comprises a male fitting.

5. The luer fitting according to claim 4, wherein the first end further comprises a threaded female fitting disposed exterior of the male fitting.

6. The luer fitting according to claim 4, wherein the male fitting comprises a longitudinal passageway extending toward the second end.

7. The luer fitting according to claim 1, wherein the first end is open.

8. The luer fitting according to claim 1, wherein the second end is closed.

9. The luer fitting according to claim 8, wherein the second end comprises a self sealing material.

10. The luer fitting according to claim 9, wherein the self sealing material comprises latex.

11. The luer fitting according to claim 1, wherein the first end comprises a luer fitting, wherein the antibacterial component is disposed within the luer fitting.

12. A luer fitting comprising a body having a first end and a second end, wherein the first end includes a stylet extending therefrom, wherein the stylet includes an antibacterial component disposed on an exterior thereof.

13. The luer fitting according to claim 12, wherein the antibacterial component comprises silver.

14. The luer fitting according to claim 12, wherein the stylet is tubular in shape.

15. The luer fitting according to claim 12, wherein the antibacterial component comprises a plurality of antibacterial components spaced along a length of the stylet.

16. The luer fitting according to claim 12, wherein the first end and the second end are in fluid communication with each other.

17. A method of reducing antibacterial activity in a catheter comprising:
providing a catheter having a lumen and a connector connected thereto;
inserting a lock solution into the lumen;
releasably affixing a luer fitting to the connector, wherein the luer fitting includes an antibacterial component; and
exposing the antibacterial component to the lock solution, wherein the antibacterial component disperses through at least a portion of the lock solution.

18. The method according to claim 17, wherein releasably affixing the luer fitting to the connector comprises inserting a stylet into the lumen, wherein the stylet includes the antibacterial component.
